(19) United States
(12) Patent Application Publication
(10) Pub. No.: US 2021/0217488 A1
(43) Pub. Date: Jul. 15, 2021

(54) FISH FARM MATERIAL HANDLING

(71) Applicant: Cryoocyte, Inc., Atlanta, GA (US)

(72) Inventors: James Webb, Jupiter, FL (US); Dmitry Kozachenok, Atlanta, GA (US)

(21) Appl. No.: 16/738,669

(22) Filed: Jan. 9, 2020

(51) Int. Cl. G16B 20/40; A01K 67/02; C12Q 1/6883; G16B 10/00; G16B 25/10; G16B 50/30; A01K 61/13

(52) U.S. Cl. CPC G16B 20/40; A01K 67/02; C12Q 1/6883; G16B 10/00; G01N 2800/50; G16B 50/30; A01K 61/13; A01K 2227/40; G16B 25/10

(57) ABSTRACT

Methods, systems, and apparatuses, including computer programs encoded on a computer-readable storage medium for fish farm material handling are described. Fish farm material includes samples collected from broodstock individuals and transported for suspension in cold storage. During the time window of cold storage, a metric of interest corresponding to the fish farm material is identified. Based on the metric of interest identification, material handling may be modified to, for example, segregate fish farm material into quarantined incubation units to inhibit infection of fish farm material within other incubation units.

### BACKGROUND

The food sector plays a significant role in being one of the main contributors to the economy of many countries, particularly in developing countries. In agriculture and aquaculture, operations management and engineering approaches have been applied to, for example, food chain logistics and sequential scheduling. Various bio-production systems and environmental compliance systems employ systems engineering perspectives with focuses on sub-systems, including optimization, supply chain systems, and IT-driven decision making for improving the efficiency, effectiveness, and sustainability of production.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

FIG. 1 is a diagram illustrating a method for implementing fish farm material handling in accordance with some embodiments

FIG. 2 is a diagram illustrating breeding decisions based on family allocation strategies in accordance with some embodiments.

FIG. 3 is a diagram illustrating breeding decisions based on disease resistance determinations in accordance with some embodiments.

FIG. 4 is a diagram illustrating breeding decisions based on analysis of progeny resulting from male-to-female pairings of breeding candidates to generate a family having a desired phenotypic makeup in the next generation in accordance with some embodiments.

FIG. 5 is a block diagram illustrating a system configured for selecting a population of broodstock individuals for use in making breeding decisions in accordance with some embodiments

### DETAILED DESCRIPTION

Gamete senescence is a phenomenon characterized by decreases in the fertility of reproductive cells and increases in the incidence of mortality in embryos arising from the nuclear fusion of pronuclei from stale gametes. For various vertebrates and invertebrates, senescence associated with aging of either or both gametes has been shown to result in a gradual loss, as a function of time, in the fertilization capacity of gametes and to support normal embryological development. For example, in-vitro and in-vivo post-ovulatory ageing of oocytes is associated with cellular changes that accompany gamete aging, such as changes in organelle and cytoskeleton distribution/organization, as well as modification of various biochemical and molecular pathways involved in cellular signaling, homeostasis, and regulation. These molecular, ultrastructural, biochemical, and/or cellular changes translate into lower fertilization rates, polyspermy, chromosomal anomalies, cell death of oocytes by apoptosis, retarded development of embryos, increased mortality rates, malformations, and other similar anomalies in development. Accordingly, preferable permutations of ageing for both oocytes and spermatozoa would be materialized by the fertilization of a fresh oocyte by a fresh spermatozoon; in contrast, less preferable permutations include fertilizing an aged or degenerating oocyte with an aged or senescent spermatozoon.

Further, although gametes aged in-vitro and in-vivo share many common properties (e.g., fertility decreases and embryonic mortality increases with increasing age of gametes), some particular traits as well as the rate or intensity of changes in processes or degradation also appear to depend on the environmental conditions to which gametes are exposed during the process of ageing. For example, spermatozoa of many marine and freshwater fish become motile upon contact with water but lose fertility and become immotile within a few minutes for many species. Due to sperm capacitation and other physiological changes, fertilizing spermatozoa must quickly enter eggs through micropyles before ova lose their fertilization competence.

Artificial reproduction under more controlled conditions has therefore become utilized as a strategy for increasing the reproductive performance of various species. However, conventional artificial reproduction technologies (even those employing hormonal therapies for inducing oocyte maturation, ovulation, and spawning) are still associated with various asynchronies in ovulation time and fry production. Accordingly, even amongst batches of gametes collected at approximately the same time (e.g., ova stripped from female fish on the same day), the quality of gametes will vary due to ageing based on differing points in time at which the individual females had ovulated and/or when the individual males had reached sexual maturity.

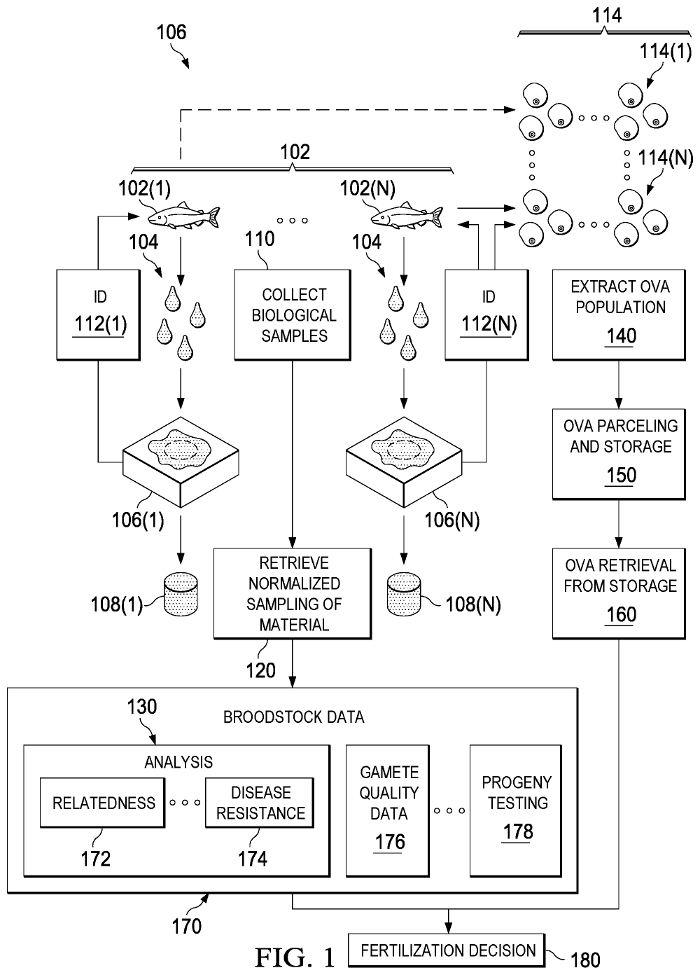

To improve the precision of fertilization timing windows and to better align periods of sperm viability with the fertile life of ova, FIGS. 1-5 describe techniques for prolongation of viable ova and/or sperm retention for improving conception rates and qualities in artificially bred animals. In some embodiments, a method of reproduction timing manipulation and production of a population of fish offspring includes obtaining and cold storing a plurality of sets of fish ova, each set of the plurality of sets of fish ova being extracted from a corresponding broodstock female. Cold storing also includes labeling each set of fish ova in relation to the corresponding broodstock female. In various embodiments, the method also includes collecting a biological sample from each broodstock female and with respect to each broodstock female associated with a corresponding set of fish ova and biological sample, after the corresponding set of fish ova has been extracted, obtaining broodstock data that includes characterization of a set of DNA sequences of interest. Subsequently, a population of sperm to fertilize the first subset of the plurality of sets of fish ova is selected based on a determination of optimizing for a breeding metric of interest. Thus, various embodiments disclosed herein provide a higher rate of improvement of desired characteristics, such as increased yield, or disease resistance, among others, which assists in sustainable agriculture and aquaculture practices.

FIG. 1 is a diagram of a method 100 for implementing fish farm material handling in accordance with some embodiments. As illustrated with respect to block 110, biological samples are collected from a plurality of a population of broodstock 102. In various embodiments, such biological samples include samples from which genetical material may be extracted. As referred to herein, the term “broodstock” (or broodfish) refers to a group of mature individuals used for breeding purposes. In some embodiments, broodstock includes a population of animals maintained in captivity as a source of replacement for, or enhancement of, seed and fry numbers in aquaculture. Although the broodstock 102 are described here in the context of broodstock females (referred to interchangeable herein as broodstock females 102) and ova, those skilled in the art will recognize that reproduction timing manipulation may be applied to the gametes of any gender or gender combination of broodstock without departing from the scope of this disclosure.

As illustrated in FIG. 1, collecting the biological samples includes collecting a sample of blood 104 from each broodstock female 102 (e.g., each of the N number of broodstock females 102 in FIG. 1, depicted as a first broodstock female 102(1) through Nth broodstock female 102(N) for ease of illustration). In some embodiments, collecting the sample of blood 104 further includes exposing blood from each of the broodstock females 102 to a respective blood booklet 106 (e.g., depicted as a first blood booklet 106(1) corresponding to the first broodstock female 102(1) through Nth blood booklet 106(N) corresponding to the Nth broodstock female 102(N) for ease of illustration). The blood booklet 106 includes one or more layers of absorbent material (not shown) for absorbing and thereby collecting the sample of blood 104 from each brood stock female. In various embodiments, the absorbent material includes any material of a porous nature, such as thick filter paper, matted cotton, cloth, other bibulous material including a mass or complex of cellulose fibers, and the like, such that the absorbent material will readily absorb or otherwise pick up a substantially constant amount of the impregnating solution (e.g., the samples of blood 104) to which it is exposed. The absorbent material is further of a porous nature such that the impregnating solution permeates substantially throughout the thickness of the absorbent material (as opposed to mere surface permeation) and is capable of retaining the absorbed, impregnating solution for use at a later point in time.

At block 120, a normalized sampling of genetic material 108 (e.g., blood) is retrieved from the blood booklet 106 associated with each individual broodstock female 102. As previously mentioned, the one or more layers of absorbent material in the blood booklet 106 absorb a substantially constant amount of blood. Accordingly, taking a similarly sized sampling from each blood booklet 106 (e.g., a hole-punched volume of the absorbent material such as illustrated in FIG. 1) yields an amount of blood cells that is substantially similar from one broodstock female 102 to another. That is, a number of blood cells in a first normalized sampling of genetic material (labeled with reference numeral 108(1) in FIG. 1 for ease of reference) for the first broodstock female 102(1) is substantially similar to a number of blood cells in a second normalized sampling of genetic material (labeled with reference numeral 108(N) in FIG. 1 for ease of reference).

The normalized samplings of genetic material 108 represents a relatively constant quantity of blood cells per hole-punched volume sample, which also equates a relatively constant level of deoxyribonucleic acid (DNA) in isolation. This decrease in sample-to-sample variation in DNA amount reduces the complexity of downstream genetics analysis 130 operations (as described in more detail below). In other embodiments, rather than collecting biological samples via blood booklets 106, the collection of biological samples at block 110 includes broodstock female tissue sampling. In various embodiments, tissue sampling includes collecting fin clips, muscle tissue plugs, and the like from each broodstock female 102.

It will be appreciated that such tissue samplings will include greater sample-to-sample variations of DNA amounts relative to the above-discussed hole-punched volumes from the blood booklets 106 due to, for example, variations in sample thicknesses depending on locations at which broodstock tissue is sampled, variations in number of cells across differing tissue locations, variations in DNA amount across differing tissue types, and the like. Accordingly, in addition to tissue sampling, these embodiments may include an additional individualization processing (not shown) to normalize the amount or concentration of DNA representing each broodstock female in advance of downstream genetics processing (e.g., downstream genetics analysis 130 of FIG. 1 and/or other operations involving sample-to-sample comparisons in which consistent DNA concentration is desirable). Additionally, in various embodiments, the blood booklets 106 may be placed into cold storage after taking the initial normalized samplings of genetic material 108. Subsequently, the blood booklets 106 operate as long-term genetic material reservoirs and may be resampled for additional genetic material as needed.

In various embodiments, the method 100 also includes tagging each broodstock female 102 with a unique identifier 112 provided by their respective blood booklets 106. For example, as illustrated in FIG. 1, a first broodstock female 102(1) is tagged with a first unique identifier 112(1) from its blood booklet 106(1). Similarly, a second broodstock female 102(N) is tagged with a second unique identifier 112(N) from its blood booklet 106(N). In this manner, a connection is created between each blood booklet 106 (and its associated samplings of genetic material) and the broodstock female 102 from which the samples of blood 104 were taken.

As illustrated with respect to block 140, a set of ova 114 are obtained via extraction from each of the population of broodstock females 102. It should be recognized that although described and illustrated here in the context of fish ova, those skilled in the art will recognize that the gametes of various non-fish species including but not limited to domesticated or wild agricultural animals, domesticated or wild aquaculture animals, shrimp, oysters, crab, lobster, mussels, other shellfish, and the like may also be processed for implementing reproduction timing manipulation without departing from the scope of this disclosure. As illustrated in FIG. 1, a first set of ova 114(1) is extracted from the first broodstock female 102(1). Similarly, a second set of ova 114(N) is extracted from the Nth broodstock female 102(N). Further, in various embodiments, the method 100 also includes tagging the set of ova 114 extracted from each broodstock female 102 with the unique identifier 112 provided by their respective blood booklets 106. For example, as illustrated in FIG. 1, the second set of ova 114(N) is tagged with the second unique identifier 112(N) from the blood booklet 106(N) corresponding to the Nth broodstock female 102(N). In this manner, a connection is created between the blood booklet 106(N) (and its associated samplings of genetic material), the set of ova 114(N), and the broodstock female 102(N) from which the ova were extracted, thereby maintaining coordination between the various operations and constituents of method 100.

At block 150, the extracted ova from block 140 are packaged into one or more storage parcels in preparation for cold storage. In various embodiments, each of the extracted ova from a single broodstock female (e.g., the second set of ova 114(N) corresponding to the Nth broodstock female 102(N)) is split or otherwise allocated across two or more different storage parcels (not shown). That is, the second set of ova 114(N) is split into smaller batches. The parceled second set of ova 114(N) may be imaged with a light table to illuminate the interior of the ova for quality determination purposes. In various embodiments, a first subset of the set of ova 114(N) (such as one or more of the two or more different storage parcels representing the extracted ova from a single broodstock female) may be utilized for testing purposes, such as for testing of ova quality for hatchery management purposes, metrics related to fertility of ova, and the like. It should be noted that although the quality determination is mentioned here with respect to the operations of block 150 prior to cold storage, those skilled in the art will recognize that the determination of ova quality may also be performed afterwards or in combination with retrieval of ova from cold storage.

After batching into the two or more different storage parcels, at least a second subset of the set of ova 114(N) (such as one or more of the two or more different storage parcels representing the extracted ova from a single broodstock female) is retained for storage purposes. In some embodiments, the at least a second subset of the set of ova 114(N) is put into cold storage within any suitable temperature-controlled environment and storable at a controlled temperature for a duration of at least twenty-four hours. In some embodiments, the at least a second subset of the set of ova 114(N) is put into cold storage within any suitable temperature-controlled environment and storable at a controlled temperature for a duration of at least one week. In various embodiments, cold storage of the ova 114 includes placing at least the second subset of the set of ova (and optionally also the first subset of the set of ova) in a refrigerated environment at a temperature substantially proximal to a freezing point of water (i.e., zero degrees Celsius) without causing freezing damage.

In general, cold storage duration is a function of storage temperature; storage duration increases as storage temperature decreases until an internal freezing point of the ova (e.g., variable depending upon the medium which the ova are in) is reached. For example, lower temperatures (i.e., closer to zero degrees Celsius) generally prolong ova shelf life (for longer than a few hours without appreciable loss in ova viability/fertility) by stabilizing and slowing the degradation of cellular bio-molecules, while also slowing the growth of any contaminant microorganisms present within or around the ova and storage medium. However, storage temperature too close to zero degrees Celsius or below zero degrees Celsius also increases risks of ova damage due to accidental freezing arising from small temperature fluctuations.

Accordingly, in one embodiment, cold storage of the ova 114 includes placing at least the second subset of the set of ova in a refrigerated environment at a temperature substantially proximal to a freezing point of water in the range of 0.0 to 1.0 degrees Celsius. In another embodiment, cold storage of the ova 114 includes placing at least the second subset of the set of ova in a refrigerated environment at a temperature proximal to a freezing point of water in the range of 1.1 to 2.0 degrees Celsius. In another embodiment, cold storage of the ova 114 includes placing at least the second subset of the set of ova in a refrigerated environment at a temperature substantially proximal to a freezing point of water in the range of 2.0 to 4.0 degrees Celsius. In other embodiments, cold storage of the ova 114 includes placing at least the second subset of the set of ova in a refrigerated environment at a temperature substantially proximal to a freezing point of water in the range of −2.0 to 0.0 degrees Celsius using super-chilling. Super-chilling includes impregnating the solution medium surrounding the target (e.g., the ova 114 for cold storage) with ions to depress the solution medium's freezing point a few, single digit degrees below zero degrees Celsius, thereby further reducing cellular degradation and microbial contaminant growth (relative to above zero degrees Celsius environments) while similarly avoiding freezing damage.

In various embodiments, cold storage of the ova 114 at block 150 includes placing at least the second subset of the set of ova (and optionally also the first subset of the set of ova), for each broodstock female 102, in a refrigerated environment for a duration of up to two weeks without a substantial drop in fertilization rates of the ova after retrieving from storage at block 160 relative to a baseline fertilization rate. For example, in some embodiments, ova 114 having approximately 90% fertilization rates on day 1 will retain the same level of fertility on days 7-10 after cold storage. Depending on variability in ova quality and the like, in other embodiments, the ova 114 may retain approximately the same levels of fertility on day 14 and beyond.

In other embodiments, cold storage of the ova 114 at block 150 includes placing at least the second subset of the set of ova (and optionally also the first subset of the set of ova), for each broodstock female 102, in a refrigerated environment for a duration of two to three weeks with approximately a 10% drop in fertilization rates of the ova after retrieving from storage at block 160 relative to a baseline fertilization rate. For example, in some embodiments, ova 114 having approximately 90% fertilization rates on day 1 will have approximately 80% fertilization rates on day 21. In this manner, cold storage of ova 114 for a time duration provides a “time window of opportunity” of a few days up to a few weeks, dependent upon acceptable subsequent fertilizability, during within which received or generated broodstock data 170 may be applied to making a breeding decision 180, such as for nucleus breeding and/or multiplication purposes. In some embodiments, the one or more of the two or more different storage parcels (representing the extracted ova from a single broodstock female) in cold storage may be packaged in a temperature-controlled vessel for transportation or shipping.

It is noted that the various operations are described here in the context of ova and cold storage for ease of description and illustration. However, it should be recognized that the operations described herein may similarly be applied to other gametes (e.g., milt from broodstock males) without departing from the scope of this disclosure. Similarly, in various embodiments, the operations described herein may similarly be applied to other stages of an organism's life cycle (e.g., embryos after fertilization of eggs) without departing from the scope of this disclosure. Further, in various embodiments, cold storage of gametes (or embryos) includes cryopreservation and storage of biological materials at extremely low temperatures (e.g., −196 degrees Celsius in liquid nitrogen). It will be appreciated that a large majority of biological activity, including the biochemical reactions that lead to cell death and DNA degradation, stops at the low temperatures associated with cold storage via cryopreservation. Accordingly, cold storage via cryopreservation extends the amount of time for which gametes and embryos may be placed in cold storage (and during which various analyses and breeding decisions may be determined).

In other embodiments, the one or more of the two or more different storage parcels (representing the extracted ova from a single broodstock female) are placed in cold storage for a period of time to generate a time window of opportunity within which received or generated broodstock data 170 may be applied to making a breeding decision 180, such as for nucleus breeding and/or multiplication purposes. By way of example and not of limitation, the broodstock data 170 includes data generated by the downstream genetics analysis of block 130. In some embodiments, the downstream genetics analysis of block 130 includes generating or receiving characterization of a set of DNA sequences of interest. For example, in various embodiments, the characterization of the set of DNA sequences of interest for one or more broodstock females 102 at block 130 includes characterization of one or more of genes, markers of genes, and/or marker(s) of a collection of genes with respect to relatedness 172 between individual broodstock females 102. The relatedness 172 of individuals may be utilized in determinizing the breeding decision 180 (e.g., developing a breeding program) such that an amount of inbreeding within a generation may be reduced to below a predetermined threshold. Further, in various embodiments, the broodstock data 170 also includes non-DNA data such as RNA-related data, protein-related data, metabolic-related data, phenotypic-data, and other data to be considered in making the breeding decision 180.

The characterization of the set of DNA sequences of interest for one or more broodstock females 102 at block 130 includes characterization of one or more of genes, markers of genes, and/or marker(s) of a collection of genes with respect to disease resistance 174. For example, in some embodiments, the characterization of the set of DNA sequences of interest includes detecting profile of a set of genes in the individual one or more broodstock females 102 indicating increased resistance to pancreas disease (e.g., infectious pancreas disease (IPD), infectious pancreatic necrosis (IPN), and the like). In other embodiments, the characterization of the set of DNA sequences of interest includes detecting profiles of a set of genes in the individual one or more broodstock females 102 indicating increased resistance to viral diseases such as infectious salmon anemia (ISA), Piscine myocarditis virus (PMCV), and the like. In various embodiments, the characterization of the set of DNA sequences of interest also includes detecting profiles of a set of genes in the individual one or more broodstock females 102 indicating increased resistance to viral diseases such as Cardiomyopathy Syndrome (CS), bacterial diseases such as Salmonid Rickettsial Syndrome (SRS), Bacterial Kidney Disease (BKD), Enteric Redmouth Disease (ERM), protozoan diseases such as Amoebic Gill Disease (AGD), metazoan diseases such as sea lice infections, and the like. Although described here in the context of genomics data with respect to relatedness 172 between individual broodstock females 102 and disease resistance 174, those skilled in the art will recognize that the characterization of a set of DNA sequences of interest at the downstream genetics analysis of block 130 may include any of various genes or genomic regions of interest (including both encoding and non-encoding regions) without departing from the scope of this disclosure. For example, in other embodiments, the characterization of a set of DNA sequences of interest may include profile detection of a set of genes in the individual one or more broodstock females 102 indicating increased resistance to other biological issues such as parasites (e.g., sea lice), chemical issues such as antibiotic sensitivity, and the like.

In various embodiments, the broodstock data 170 also includes gamete quality data 176 and progeny testing data 178. By way of example and not of limitation, the broodstock data 170 includes generating or receiving gamete quality data 176 that includes one or more of ova quality determinations (such as mentioned in relation to block 150) and sperm quality determinations. Additionally, in various embodiments, the broodstock data 170 also includes generating or receiving progeny testing data 178 associated with future generations resulting from mating schemes corresponding to the broodstock females 102 (i.e., the previous generation).

In various embodiments, upon retrieval of the ova from cold storage at block 160, one or more of the broodstock data 170 is applied in making a breeding decision 180, such as for nucleus breeding and/or multiplication purposes. Such breeding decisions 180 may include, for example, mating schemes with specific male-and-female pairings, family allocation decisions, family isolation decisions, family batching decisions, gamete elimination decisions, and the like. The breeding decision 180 includes selecting, based at least in part on the broodstock data 170, a first subset of the ova retrieved from cold storage at block 160 to be fertilized. In various embodiments, the breeding decision 180 also includes selecting a population of sperm (e.g., from a single male or multiple males) to fertilize the ova retrieved from cold storage based on a determination of optimizing for a breeding metric of interest.

For example, in some embodiments, the selecting of the population of sperm from one or more males for fertilization of the ova retrieved from cold storage (associated with one or more broodstock females 102) is determined for producing a population of fish offspring having a desired phenotype of interest or probabilities of observing the desired phenotype of interest in offspring resulting from combination of the selected population of sperm and the ova retrieved from cold storage. It should be recognized that although various embodiments are described here in the context of screening for phenotypes and/or genotypes in the context of disease resistance, various breeding metrics of interest other than those conferring disease may also be selected for without departing from the scope of this disclosure. By way of non-limiting example, such breeding metrics of interest may include growth characteristics, harvest traits, early or late maturation age, and the like.

As used herein, a “phenotype” refers to certain observable characteristic or trait of an organism, such as morphological, developmental, biochemical, physiological, or behavioral properties. In various embodiments, adult size, flesh color, flesh quality, gender, growth rates, filet characteristics, resistance to environmental stresses, risk of developing certain types of diseases, late maturation (e.g., age at sexual maturation), and the like are examples of phenotypes. As used herein, “genotype” refers to information pertaining to the genetic constitution of a cell, an organism, or an individual in reference to a specific character under consideration, for example but not limited to, information pertaining to a combination of alleles located on homologous chromosomes that is associated with specific characteristics or traits.

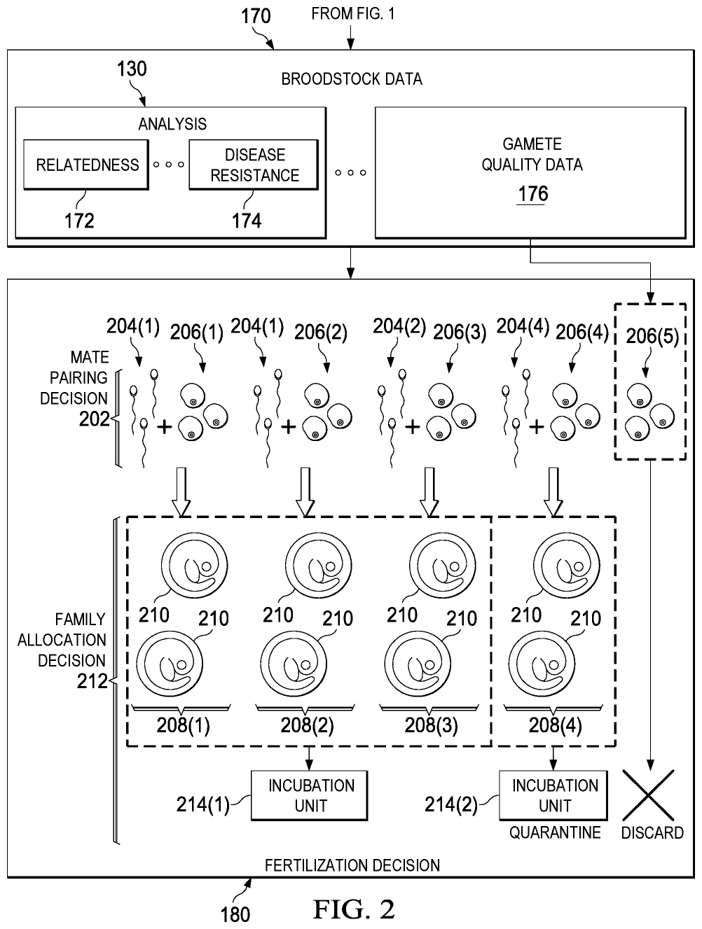

As described in more detail below with respect to FIGS. 2-5, the cold storage of gametes associated with blocks 140-160 provides an inventoried library of female gametes (e.g., at block 150) and male gametes (not shown). The time interval within which gametes are placed in cold storage provides a time window of opportunity within which broodstock data 170 may be obtained (i.e., a period of time that would otherwise not be available due to the normal time-scales of gamete degradation, such as due to gamete senescence and the like, when extended cold storage is unavailable). The broodstock data 170 may thus be utilized in making a breeding decision 180 to produce a population of progeny having desirable characteristics.

Referring now to FIG. 2, illustrated is a diagram showing breeding decisions based on family allocation strategies in accordance with some embodiments. In various embodiment, breeding decisions (e.g., breeding decision 180 of FIG. 1) include determining mate pairing decision(s) 202 including the application of milt from a specific male to the ova of a specific female, with the resulting progeny of that particular mate pairing being a “family”. As illustrated in FIG. 2, a first population of sperm 204(1) from a first broodstock male (not shown) fertilizes a first set of ova 206(1) from a first broodstock female (e.g., first broodstock female 102(1) of FIG. 1) to produce a first family 208(1) including one or more progeny individuals 210. In various embodiments, the first population of sperm 204(1) from the first broodstock male also fertilizes at least a second set of ova 206(2) from a second broodstock female different from the first broodstock female (e.g., a Nth broodstock female 102(N) of FIG. 1) to produce a second family 208(2) including one or more progeny individuals 210. In this manner, the first population of sperm 204(1) from a single broodstock male fertilizes the ova from multiple broodstock females to produce multiple different families.

Similarly, in various embodiments, the set of ova (e.g., the first set of ova 114(1) extracted from the first broodstock female 102(1) of FIG. 1) extracted from a single broodstock female may be grouped into different subsets, with each different subset fertilized by a different broodstock male. As illustrated in FIG. 2, a second population of sperm 204(2) from a second broodstock male (not shown) fertilizes a third set of ova 206(3) (i.e., the third set of ova different from the first set of ova) from the first broodstock female (e.g., first broodstock female 102(1) of FIG. 1) to produce a third family 208(3) including one or more progeny individuals 210. In this manner, the ova from a single broodstock female (i.e., the first set of ova 206(1) and the third set of ova 206(3) as illustrated in FIG. 2) are respectively fertilized by sperm from multiple broodstock males to produce multiple different families.

Additionally, as illustrated in FIG. 2, a fourth population of sperm 204(4) fertilizes a fourth set of ova 206(4) to produce a fourth family 208(4) including one or more progeny individuals 210. Accordingly, as described herein, the various differentiated families 208 may be formed by mate pairings including single male with multiple females, single female with multiple males, single male with single female, or any combination thereof.

In some embodiments, such as for nucleus breeding, numerous differentiated families 208 are produced from selected broodstock. The broodstock utilized are often chosen based on individual performance of the broodstock individuals and/or performance of their respective families 208 to advance an average performance of the next generation's breeding population (i.e., the progeny individuals 210 become potential broodstock individuals for the subsequent generation). Performance, in this context, refers to quantitative results of measured traits (e.g., growth, disease resistance, fillet characteristics, and the like). In various embodiments, the mate pairing decisions 202 is determined based at least in part on broodstock data 170 including genomics data with respect to relatedness 172 (and therefore levels of inbreeding) between broodstock individuals, disease resistance 174, gamete quality data 176 associated with the broodstock individuals, and the like. In nucleus breeding, families 208 are generally managed independently to preserve family-line differentiated genetics and therefore family allocation strategies generally are not applied.

In contrast to nucleus breeding, ova production for multiplication purposes is a linear process in which multiplied ova are grown for harvesting purposes and do not enter the next generation of nucleus breeding. Accordingly, relatedness, inbreeding, and familial differentiation are not of principle importance. However, in various embodiments, determination of the mate pairing decisions 202 for multiplication is also similarly determined based at least in part on broodstock data 170 including genomics data with respect to relatedness 172 (and therefore levels of inbreeding) between broodstock individuals, disease resistance 174, gamete quality data 176 associated with the broodstock individuals, and the like. The broodstock employed are principally chosen based on the performance of their respective families 208 and/or individual performance of the broodstock individuals, but numerous undifferentiated families are produced for use as production fish in commercial farming environments.

In various embodiments, optimizing for a breeding metric of interest includes a determination of allocating progeny resulting from fertilizing ova of one or more broodstock females to a single incubation unit (also generally referred to as “family batching”). In one embodiment, the breeding decision 180 includes making a family allocation decision 212 along with mating decisions by batching the families 208(1), 208(2), and 208(3) into a single, first incubation unit 214(1). In some embodiments, the family allocation decision 212 is determined based on, for example, an identification of broodstock females homozygous for a breeding metric of interest (e.g., a characteristic, phenotypic trait, and the like) using genomics data from genetics analysis 130 and batching the resulting progeny individuals 210 into a singular incubation unit 214 for a specific customer. In other embodiments, the family allocation decision 212 is determined based on, for example, genomics data from genetics analysis 130 including relatedness 172 (and therefore levels of inbreeding) between broodstock individuals to equalize (or otherwise decrease differences between) familial representation amongst multiple incubator units 214 and/or development of a pedigree-based mating strategies using broodstock data 170.

Although described here in the context of allocating the families 208 of multiple broodstock males and multiple broodstock females into the first incubation unit 214(1), it should be recognized that family allocation decisions may include any number of families 210 resulting from any number of broodstock males and/or broodstock females. For example, in some embodiments, the family allocation decision 212 includes allocating multiple families with progeny individuals 210 resulting from a single broodstock male into a single incubation unit. In other embodiments, the family allocation decision 212 includes allocating multiple families with progeny individuals 210 resulting from a single broodstock female into a single incubation unit.

In various embodiments, the family allocation decision 212 includes quarantine decisions or elimination/discard decisions. The operations of collecting the biological samples at block 110 or extracting ova populations at block 140 of FIG. 1 also includes collecting a volume of ovarian fluid from one or more broodstock females 102. The family allocation decision 212 also includes determining, based at least in part on broodstock data 170 including genomics data with respect to biological samples (e.g., gametes, tissue samples, blood samples, ovarian fluids, and the like) collected from broodstock individuals, the prevalence or a likelihood of vertically-transmitted diseases (as opposed to horizontal transmission which refers to transmission of infections or diseases between members of the same species that are not in a parent-child relationship). For example, in some embodiments, the family allocation decision 212 also includes screening of ovarian fluid ribonucleic acid (RNA) for the presence of vertically transmitted disease. In various embodiments, the family allocation decision 212 also includes screening of ovarian fluid DNA for the presence of bacterial diseases and DNA genome viruses. In one embodiment, based on detecting viral RNA via reverse transcriptase polymerase chain reaction (RT-PCR) techniques indicating the presence and therefore a risk of virus transmission, the fourth family 208(4) produced by a broodstock female having virus-positive ovarian fluid is quarantined and separately batched into a different, second incubation unit 214(2) and therefore isolated from other incubation units 214.

In various embodiments, the family allocation decision 212 also includes determining, based at least in part on broodstock data 170 including gamete quality data 176, a measure of gamete quality (e.g., genetics-based or otherwise). For example, in one embodiment, detecting the presence of maturation proteins in ovarian fluids is indicative of poor quality ova that would result in a reduction of embryo survival rates. Accordingly, a fifth set of ova 206(5) extracted from a broodstock female having maturation-protein-positive ovarian fluid is discarded instead of being fertilized. Similarly, ova quality may be determined based on visual inspection with poor quality eggs (e.g., over- and/or under-maturation) being discarded instead of being fertilized. In various embodiments, ova quality may be determined based on ovarian fluid pH measurements. In various embodiments, similar gamete quality analyses are performed with respect to broodstock males with poor quality sperm (e.g., having low motility and/or low density) being discarded instead of being used to fertilize ova. Further, in various embodiments, gamete quality analyses include screening for the presence of bacterial diseases such as SRS, BKD, and the like. Thus, as described herein, genotyping (and also phenotypic analysis) allows for the determining of improved mate pairing decisions 202 based on, for example but not limited to, assessments of relatedness and disease prevalence within broodstock populations.

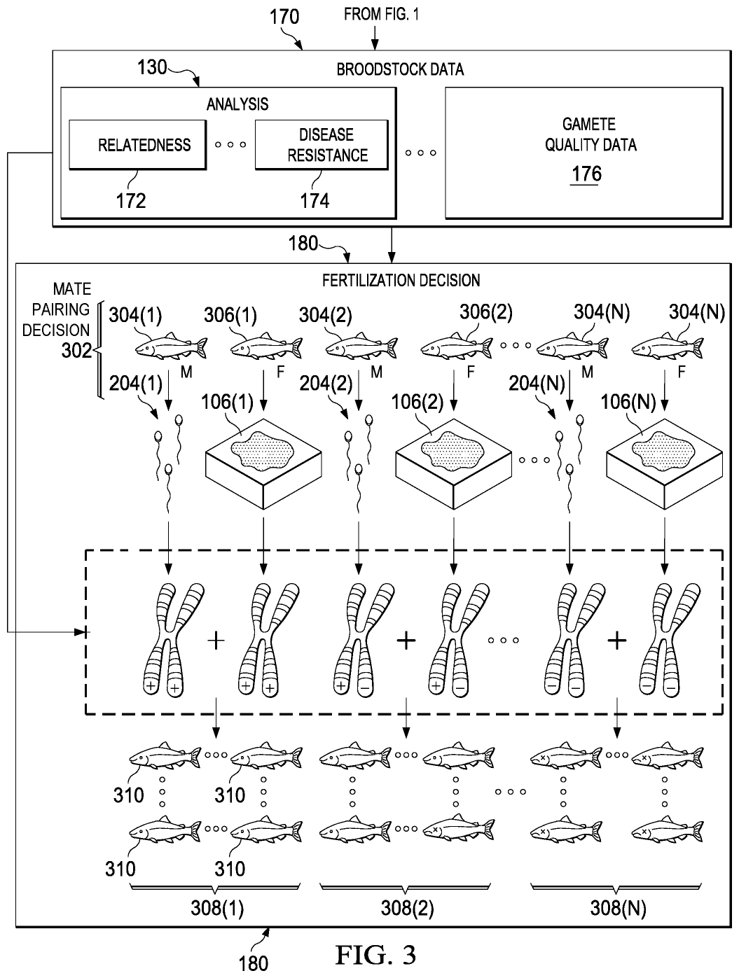

Referring now to FIG. 3, illustrated is a diagram showing breeding decisions based on disease resistance determinations in accordance with various embodiments. In addition to the disease screening of female broodstock ovarian fluids to determine whether ova should be discarded (or quarantined) as previously discussed with respect to FIG. 2, in various embodiments, optimizing for a breeding metric of interest includes optimizing for a disease resistance characteristic.

In particular, FIG. 3 is a diagram illustrating making breeding decisions (e.g., breeding decision 180 of FIG. 1) which include determining mate pairing decision(s) 302 including the application of milt from a specific male to the ova of a specific female, with the resulting progeny of that particular mate pairing being a “family”, such that a portion of the resulting progeny have a desired disease resistance. In various embodiments, making a breeding decision includes selecting broodstock individuals for mating based on disease resistance characteristics of the broodstock individuals and/or anticipated disease resistance characteristics of their respective families to advance an average disease resistance performance of the next generation.

A mate pairing decision 302 is determined based at least in part on broodstock data 170 including genomics data with respect to biological samples (e.g., gametes, tissue samples, blood samples, and the like) collected from broodstock individuals (such as previously discussed in more detail with respect to blocks 120, 130, and/or 140 of FIG. 1). For example, in various embodiments, the family allocation decision includes assessing broodstock resistance of one or more broodstock males 304 and/or broodstock females 306 to a disease (e.g., infectious pancreas disease (IPD), infectious pancreatic necrosis (IPN), and the like). Further, in various embodiments, broodstock resistance to diseases is assessed based on the presence of putative causal single nucleotide polymorphisms (SNPs) (either positively known or hypothesized to be causally linked) associated with resistance to each of one or more diseases.

As referred to herein, the terms “genetically analyzing” or “genetic analysis” refer to both genotypic- and phenotypic-assessments, individually or in combination. For example, in various embodiments, genetically analyzing the biological samples collected from broodstock individuals includes characterizing a set of DNA sequences of interest. In some embodiments, genetic analysis by characterizing a set of DNA sequences of interest includes characterizing non-coding regions of DNA. In other embodiments, genetic analysis by characterizing a set of DNA sequences of interest includes characterizing a set of genes related to disease resistance.

For example, genetically analyzing a first population of sperm 204(1) from a first broodstock male 304(1) and a first blood booklet 106(1) containing blood from a first broodstock female 306(1) includes determining the SNP genotypes of broodstock individuals through polymerase chain reaction (PCR) techniques using one or more disease-specific primer/probe sets. As illustrated, the first broodstock male 304(1) is determined based on its SNP genotype to be homozygous dominant (i.e., containing two copies of the dominant gene) in its resistance to a given disease (e.g., any of IPN, PD, and the like corresponding to the disease-specific primer set used for SNP genotyping). Similarly, the first broodstock female 306(1) is also determined based on its SNP genotype to be homozygous dominant in its resistance to the given disease. Accordingly, a hypothetical first family 308(1) arising from a mate pairing decision 302 including the first broodstock male 304(1) and the first broodstock female 306(1) would include progeny individuals 310 that are all also homozygous dominant in their resistance to the given disease.

In various embodiments, genetically analyzing a second population of sperm 204(2) from a second broodstock male 304(2) and a second blood booklet 106(2) containing blood from a second broodstock female 306(2) includes determining the SNP genotypes of broodstock individuals through PCR techniques using one or more disease-specific primer/probe sets. As illustrated, the second broodstock male 304(2) is determined based on its SNP genotype to be heterozygous (i.e., having two different alleles of a particular gene, such as by having one copy of the dominant gene and one copy of the recessive gene) in its resistance to the given disease. Similarly, the second broodstock female 306(2) is also determined based on its SNP genotype to be heterozygous in its resistance to the given disease. Accordingly, a hypothetical second family 308(2) arising from a mate pairing decision 302 including the second broodstock male 304(2) and the second broodstock female 306(2) would generally include a mixture of progeny including some progeny individuals 310 that are homozygous dominant, some progeny individuals 310 that are heterozygous, and/or some progeny individuals 310 that are homozygous recessive in their resistance to the given disease.

Further, in various embodiments, genetically analyzing a N-th population of sperm 204(N) from a Nth broodstock male 304(N) and a N-th blood booklet 106(N) containing blood from a N-th broodstock female 306(N) includes determining the SNP genotypes of broodstock individuals through PCR techniques using one or more disease-specific primer/probe sets. As illustrated, the N-th broodstock male 304(N) is determined based on its SNP genotype to be homozygous recessive (i.e., containing two copies of the recessive gene) in its resistance to a given disease (e.g., any of IPN, PD, and the like corresponding to the disease-specific primer set used for SNP genotyping). Similarly, the N-th broodstock female 306(N) is also determined based on its SNP genotype to be homozygous dominant in its resistance to the given disease. Accordingly, a hypothetical N-th family 308(N) arising from a mate pairing decision 302 including the N-th broodstock male 304(N) and the N-th broodstock female 306(N) would include progeny individuals 310 that are all also homozygous recessive in their resistance to the given disease.

Accordingly, in various embodiments, a mate pairing decision 302 is determined based at least in part on broodstock data 170 including genomics data with respect to characterization of one or more of genes, markers of genes, and/or marker(s) of a collection of genes with respect to disease resistance 174. In various embodiments, genotyping of biological samples to determine disease resistance 174 provides a characterization (e.g., a profile) of the set of genes associated with resistance to a given disease in the broodstock individuals as described above.

In various embodiments, the characterization of the set of genes serves as the basis for ranking the broodstock individuals (or broodstock pairings) into a plurality of ranking groups, each ranking group having a different threshold or range of disease-resistance characterizations. For example, in the context of FIG. 3, the first broodstock male 304(1) or the first broodstock female 306(1) (or a mate pairing decision 302 including both the first broodstock male 304(1) and the first broodstock female 306(1)) is ranked into a first ranking group based on the broodstock individual(s) being homozygous dominant in its resistance to a given disease. Accordingly, all progeny individuals 310 resulting from mate pairing decisions 302 including homozygous dominant parents will also be homozygous dominant in their resistance to the given disease.

Additionally, the second broodstock male 304(2) or the second broodstock female 306(2) (or a mate pairing decision 302 including both the second broodstock male 304(2) and the second broodstock female 306(2)) is ranked into a second ranking group based on the broodstock individual(s) being heterozygous in its resistance to the given disease. Accordingly, mate pairing decisions 302 including heterozygous parents (e.g., the second family 308(2)) will result in a mixture of progeny individuals 310 having differing genetic profiles for disease resistance relative to the given disease. In particular, mate pairing decisions 302 including heterozygous parents would result in an approximate 1:2:1 ratio mixture of progeny individuals 310 that are homozygous dominant:heterozygous:homozygous-recessive in their resistance to the given disease, respectively.

FIG. 3 also shows the N-th broodstock male 304(N) or the N-th broodstock female 306(N) (or a mate pairing decision 302 including both the N-th broodstock male 304(N) and the N-th broodstock female 306(N)) being ranked into a third ranking group based on the broodstock individual(s) being homozygous recessive in its resistance to the given disease. Accordingly, all progeny individuals 310 resulting from mate pairing decisions 302 including homozygous recessive parents will also be homozygous recessive in their resistance to the given disease.

In embodiments for which optimizing for a breeding metric of interest includes optimizing for a population of offspring having a desired resistance to the given disease (i.e., one or more families having a desired phenotypic makeup), the first ranking group having homozygous dominant broodstock individuals (or pairings of homozygous dominant broodstock individuals) is preferable over the second ranking group having heterozygous broodstock individuals (or pairings of heterozygous broodstock individuals) because mate pairings within the first ranking group have a higher probability (or in raw quantity terms, a greater number of progeny individuals) of resulting in progeny having the desired disease resistance relative to mate pairings within the second ranking group. Similarly, the first ranking group having homozygous dominant broodstock individuals (or pairings of homozygous dominant broodstock individuals) is preferable over the third ranking group having homozygous recessive broodstock individuals (or pairings of homozygous recessive broodstock individuals) when breeding for disease resistance. Additionally, the second ranking group having heterozygous broodstock individuals (or pairings of heterozygous broodstock individuals) is also preferable over the third ranking group having homozygous recessive broodstock individuals (or pairings of homozygous recessive broodstock individuals) when breeding for disease resistance.

It should be noted that although the determining of breeding decisions based on disease resistance screening is described with respect to FIG. 3 in the context of a single disease and one broodstock individual for each gender within the three different ranking groups, various numbers of broodstock individuals, mate pairing decisions, numbers of diseases to be screened for, numbers of ranking groups, and the like may be implemented without departing from the scope of this disclosure.

For example, in some embodiments and with continued reference to FIG. 3, a fourth broodstock male being homozygous dominant in its resistance for the given disease (not shown) and a fourth broodstock female being heterozygous in its resistance for the given disease (not shown) are ranked into a fourth ranking group. Similarly, in various embodiments, a fifth broodstock male being homozygous recessive in its resistance for the given disease (not shown) and a fifth broodstock female being heterozygous in its resistance for the given disease (not shown) are ranked into a fifth ranking group. Accordingly, mate pairing decisions corresponding to the fourth ranking group (and/or the fifth ranking group) generates a mixture of progeny individuals having differing genetic profiles for disease resistance relative to the given disease. In particular, mate pairing decisions corresponding to the fourth ranking group would result in an approximate 3:1 ratio mixture of progeny individuals 310 that are homozygous dominant:heterozygous in their resistance to the given disease, respectively. Similarly, mate pairing decisions corresponding to the fifth ranking group would result in an approximate 3:1 ratio mixture of progeny individuals 310 that are homozygous recessive:heterozygous in their resistance to the given disease, respectively.

In various embodiments, the determining of breeding decisions based on disease resistance screening of FIG. 3 includes genotyping biological samples of broodstock individuals to determine genetic profiles with respect to two or more diseases. Accordingly, it will be appreciated that the number of ranking groups may exponentially grow based on disease resistance screening with increasing quantities of gene sets under consideration. Further, it should be understood that although disease resistance and screening is described here in the context of diploid organisms and a single gene having two alleles for each of description and illustration, those skilled in the art will recognize that the disease resistance screening concepts described here may be applied to organisms having any combination of ploidy, interaction between multiple genes, allelic variations, and the like without departing from the scope of this disclosure.

In some embodiments, genotyping of biological samples collected from broodstock individuals includes, but is not limited to: genotypic analysis of DNA isolated from male broodstock sperm for the presence of SNP markers related to disease susceptibility, genotypic analysis of DNA isolated from broodstock individuals for determining parentage and reduction of inbreeding, assessment of mRNA expression of transferrin in RNA isolated from broodstock mucous as a correlation of sea lice infection, proteomic analysis of liver tissue to determine whether broodstock individuals have been administered feed with genetically modified ingredients, and the like. In this manner, genotyping to provide an assessment of parental resistance to disease (or genotyping with respect to other breeding metrics of interest) allows for improved and/or more precise mate pairing decisions 302. In particular, genotyping allows for selection of mate pairings that provide for higher rates of improvement of desired characteristics in the resulting progeny population, such as disease resistance (FIG. 3) or increased yield (not shown). Further, determining of breeding decisions based on genotyping increases the precision of breeding-generated phenotypes and/or the breadth of accessible genetic variations.

Additionally, in other embodiments, genotyping of biological samples collected from broodstock individuals includes parentage analysis using microsatellite markers. Although various embodiments are described here in the context of SNP analyses, those skilled in the art will recognize that microsatellite markers may be similarly used in assessing parentage. For example, microsatellite analysis including the selecting/screening for of 3-4 microsatellite markers provides an economical alternative matching the resolving power of 50+ SNP markers. Such parentage analyses may, in various embodiments, be used in the minimization of inbreeding between mating pairs, the design of offspring populations through optimum contribution selection, and in developing a trackable product.

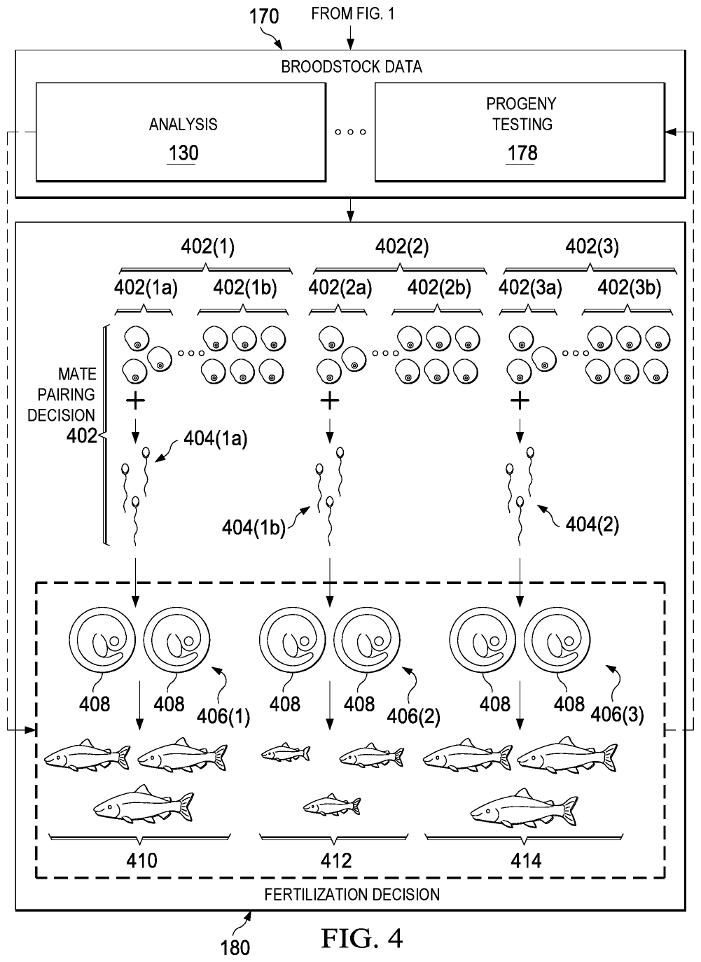

Referring now to FIG. 4, illustrated is a diagram illustrating breeding decisions based on analysis of progeny resulting from male-to-female pairings of breeding candidates to generate a family having a desired phenotypic and/or genotypic makeup in the next generation in accordance with some embodiments. In addition to the genotyping of biological samples collected from broodstock individuals such as previously discussed with respect to FIG. 3, in various embodiments, optimizing for a breeding of metric includes a progeny-based analysis for making breeding decisions.

In particular, FIG. 4 is a diagram illustrating making breeding decisions (e.g., breeding decision 180 of FIG. 1) by genetically analyzing progeny populations (e.g., embryos) resulting from various fertilizations amongst breeding candidate gametes before determining mate pairing decision(s) for selected breeding individuals. Further, the breeding decision also includes determining mate pairing decision(s) for selected breeding individuals including the application of milt from a specific male to the ova of a specific female, with the resulting progeny of that particular mate pairing being a “family” having a desired phenotypic makeup, such as related to growth rate, adult size, disease resistance, and the like. In various embodiments, the breeding decision also includes determining mate pairing decision(s) for selected breeding individuals including the application of milt from a specific male to the ova of a specific female, with the resulting progeny of that particular mate pairing being a family having a desired genotypic makeup, as embryo selection (in contrast to the phenotypic-based analysis of fertilization and cleavage symmetry) is based on genotypic markers of non-additive and epigenetic variation.

As shown, a first set of ova 402(1) includes a plurality of ova having been extracted from a first broodstock female (not shown but in a manner similar to the relationship between broodstock females 102 and sets of ova 114 of FIG. 1). A second set of ova 402(2) includes a plurality of ova having been extracted from a second broodstock female (not shown) and a third set of ova 402(3) includes a plurality of ova having been extracted from a third broodstock female (not shown).

The first set of ova 402(1) is segmented into a first subset of the first set of ova 402(1a) and at least a second subset of the first set of ova 402(1b). In some embodiments, the first set of ova 402(1) may be grouped into three or more subsets of ova. The second set of ova 402(2) is segmented into a first subset of the second set of ova 402(2a) and at least a second subset of the second set of ova 402(2b). In some embodiments, the second set of ova 402(2) may be grouped into three or more subsets of ova. The third set of ova 402(3) is also segmented into a first subset of the third set of ova 402(3a) and at least a second subset of the third set of ova 402(3b). In some embodiments, the third set of ova 402(3) may be grouped into three or more subsets of ova.

In one embodiment, a first population of sperm from a first broodstock male 404(1a) fertilizes the first subset of the first set of ova 402(1a) from a first broodstock female (e.g., first broodstock female 102(1) of FIG. 1) to produce a first family 406(1) including multiple progeny individuals 408. In various embodiments, the first family 406(1) includes two or more embryos arising from combination of gametes from the first broodstock male and the first broodstock female (i.e., the sets of ova 402(1a) and sperm 404(1a)). Although primarily discussed here only in the context of embryos, those skilled in the art will recognize that progeny-based analysis may be performed at any period of an organism's life cycle starting from a single-celled zygote after fertilization and onwards, including cleavage periods, blastula periods, hatching periods, larval periods, fetal periods (in embodiments utilizing, for example, agricultural animals), and the like. In some embodiments, zygotic progeny individuals 408 may be optionally cultured to give rise to more than one cell.

In various embodiments, the first family 406(1) is analyzed genetically (including genotypically, for example) or epigenetically as described herein utilizing cell, molecular, microscopic methods, and the like to evaluate the genotypes of the progeny individuals 408 for generating progeny testing data 178. Genetic analysis 130 of the first family 406(1) evaluates genotypes of the progeny individuals 408 and determines via quantitative trait locus (QTL), SNP, and/or genetic marker analyses related to growth and/or adult size that a population of offspring arising from the breeding candidates (i.e., the first broodstock male and the first broodstock female corresponding to the first family 406(1)) would grow into an adult population at time=t having a size distribution 410 as illustrated in FIG. 4.

Similarly, a second population of sperm from the first broodstock male 404(1b) fertilizes the first subset of the second set of ova 402(2a) from a second broodstock female to produce a second family 406(2) including multiple progeny individuals 408. In various embodiments, the second family 406(2) includes two or more embryos arising from combination of gametes from the first broodstock male and the second broodstock female (i.e., the sets of ova 402(2a) and sperm 404(1b)). The second family 406(2) is analyzed genetically (including genotypically, for example) or epigenetically as described herein utilizing cell, molecular, microscopic methods, and the like to evaluate the genotypes of the progeny individuals 408 for generating progeny testing data 178. Genetic analysis 130 of the second family 406(2) evaluates genotypes of the progeny individuals 408 and determines via quantitative trait locus (QTL), SNP, and/or genetic marker analyses related to growth and/or adult size that a population of offspring arising from the breeding candidates (i.e., the first broodstock male and the second broodstock female corresponding to the second family 406(2)) would grow into an adult population at time=t having a size distribution 412 as illustrated in FIG. 4.

A first population of sperm from the second broodstock male 404(2) fertilizes the first subset of the third set of ova 402(3a) from a third broodstock female to produce a third family 406(3) including multiple progeny individuals 408. In various embodiments, the third family 406(3) includes two or more embryos arising from combination of gametes from the second broodstock male and the third broodstock female (i.e., the sets of ova 402(3a) and sperm 404(2)). The third family 406(3) is analyzed genetically (including genotypically, for example) or epigenetically as described herein utilizing cell, molecular, microscopic methods, and the like to evaluate the genotypes of the progeny individuals 408 for generating progeny testing data 178. Genetic analysis 130 of the third family 406(2) evaluates genotypes of the progeny individuals 408 and determines via quantitative trait locus (QTL), SNP, and/or genetic marker analyses related to growth and/or adult size that a population of offspring arising from the breeding candidates (i.e., the second broodstock male and the third broodstock female corresponding to the third family 406(3)) would grow into an adult population at time=t having a size distribution 414 as illustrated in FIG. 4.

Following genetic analysis 130 of the various families 406, making a breeding decision includes selecting breeding candidates for mating (e.g., any of the first and second broodstock males and first through third broodstock females, subsequently referred to as selected breeding individual(s)) to optimize for a breeding metric of interest. In the context of adult size (and therefore increased production yield) being the metric of interest in FIG. 4, optimizing for the breeding metric of interest includes selecting mate pairings determined to give rise to families having a higher average adult weight in the next generation. For example, as illustrated in FIG. 4, selecting the first broodstock male and the first broodstock female for mating is determined to result in a size distribution 410 that is greater (e.g., larger/heavier) relative to a size distribution 412 that would arise from a mate pairing of the same first broodstock male and the second broodstock female. Similarly, selecting the second broodstock male and the third broodstock female for mating is determined to result in a size distribution 414 that is greater relative to the size distribution 412 that would arise from a mate pairing of the same first broodstock male and the second broodstock female.

When optimizing for the breeding metric of interest being adult size, selecting the first broodstock male and the first broodstock female for mating is preferable over a pairing of the first broodstock male and the second broodstock female. Accordingly, the second subset of the first set of ova 402(1b) (which is stored in cold storage from block 150 of FIG. 1 for at least a portion of the time during performing of FIG. 4's progeny-based genetics analysis) is subsequently retrieved from cold storage and fertilized with sperm from the first broodstock male.

In various embodiments, optimizing for the breeding metric of interest also includes gamete selection based on genetic calculations. Gamete genetic information such as genome sequences and/or marker information is obtained and stored. In various embodiments, making a breeding decision includes making a specification of one or more phenotypes and/or genotypes of interest in hypothetical offspring (i.e., the progeny generation as described herein). For example, a single marker genotype confers a phenotype for IPN resistance. Statistical information pertaining to the likelihood of observing phenotypes or genotypes of interest are determined, based on the genotypes of the different breeding candidates and/or their progeny individuals resulting from one or more test crosses of the breeding candidates. For example, probabilities of the phenotypes of interest in the progeny population resulting from different broodstock male-female combinations are computed. Based on the statistical information, one or more breeding individuals are selected for mating.

Although described with respect to FIG. 4 in the specific context of progeny-based genetics analysis to select for growth characteristics, those skilled in the art will recognize that any breeding metric of interest (or combinations of metrics) may serve as the basis for selecting breeding individuals and determining mate pairing decisions. For example, in various embodiments, progeny-based genetics analysis includes genetically analyzing the progeny individuals 408 to identify pairings of breeding candidates (e.g., male and female broodstock individuals as parents of the progeny individuals 408) that would give rise to a progeny population having increased disease resistance (as opposed to or optionally in addition to genetic analysis of gametes or other biological samples collected from the parent generation such as previously discussed with respect to FIG. 3). In other embodiments, progeny-based genetics analysis includes genetically analyzing the progeny individuals 408 to identify pairings of breeding candidates that would give rise to a progeny population calculated to have a distribution of progeny individuals having a desired phenotypic and/or genotypic makeup, such as related to one or more quantitative results of measured traits (e.g., growth, disease resistance, fillet characteristics, and the like), to advance an average performance of the next generation with respect to one or more characteristics of interest.

Additionally, in various embodiments, progeny-based genetics analysis includes, but is not limited to: analyzing half-sib embryo families using SNP chips for determination of recombination frequencies and estimates of non-additive variation, assessment of mRNA expression of parental-specific mono-allelic genes imprinted into embryo development as a marker of epigenetic variation, monitoring developmental changes in yet-to-be identified marker protein concentrations in embryos as an indicator of long-term survival, and the like. In this manner, the progeny-based genetics analysis and gamete storage for reproduction timing manipulation as described herein provides for improving one or more characteristics of progeny/offspring (e.g., genetic or epigenetic modification) and includes more precise selection of desirable characteristics to be introduced into progeny populations.

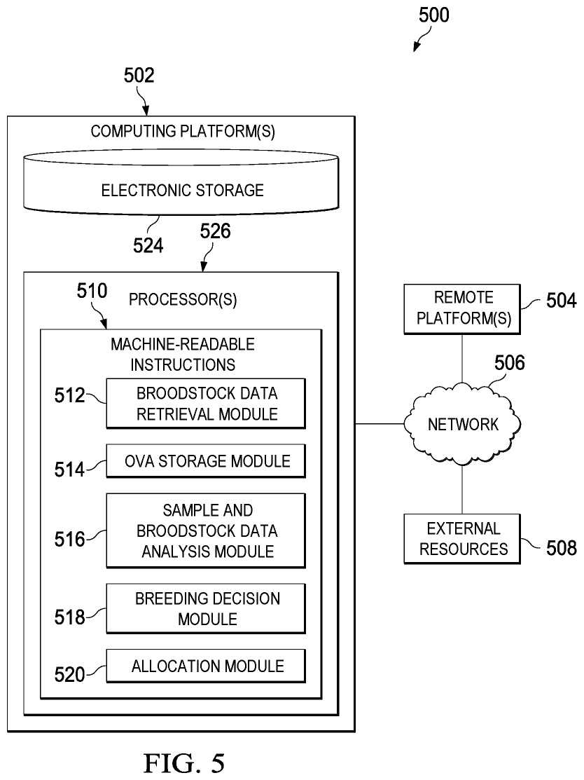

FIG. 5 is a block diagram illustrating a system 500 configured to perform selection of a population of broodstock individuals for use in making breeding decisions in accordance with some embodiments. In some embodiments, the system 500 includes one or more computing platforms 502. The computing platform(s) 502 may be configured to communicate with one or more remote platforms 504 according to a client/server architecture, a peer-to-peer architecture, and/or other architectures via a network 506. Remote platform(s) 504 may be configured to communicate with other remote platforms via computing platform(s) 502 and/or according to a client/server architecture, a peer-to-peer architecture, and/or other architectures via the network 506. Users may access system 500 via remote platform(s) 504.

In some implementations, computing platform(s) 502, remote platform(s) 104, and/or external resources 508 may be operatively linked via one or more electronic communication links. For example, such electronic communication links may be established, at least in part, via the network 506 including networks such as the Internet and/or other networks. It will be appreciated that this is not intended to be limiting, and that the scope of this disclosure includes implementations in which computing platform(s) 502, remote platform(s) 504, and/or external resources 508 may be operatively linked via some other communication media.

A given remote platform 504 may include one or more processors configured to execute computer program modules. The computer program modules may be configured to enable an expert or user associated with the given remote platform 504 to interface with system 500 and/or external resources 508, and/or provide other functionality attributed herein to remote platform(s) 504. By way of non-limiting example, a given remote platform 504 and/or a given computing platform 502 may include one or more of a server, a desktop computer, a laptop computer, a handheld computer, a tablet computing platform, a NetBook, a Smartphone, a gaming console, and/or other computing platforms. External resources 508 may include sources of information outside of system 500, external entities participating with system 500, and/or other resources. In some implementations, some or all of the functionality attributed herein to external resources 508 may be provided by resources included in system 500.

In various embodiments, the computing platform(s) 502 are configured by machine-readable instructions 510 including one or more instruction modules. In some embodiments, the instruction modules include computer program modules. The instruction modules include one or more of a broodstock data retrieval module 512, an ova storage module 514, a sample and broodstock data analysis module 516, a breeding decision module 518, and an allocation module 520. Each of these modules may be implemented as one or more separate software programs, or one or more of these modules may be implemented in the same software program or set of software programs. Moreover, while referenced as separate modules based on their overall functionality, it will be appreciated that the functionality ascribed to any given model may be distributed over more than one software program. For example, one software program may handle a subset of the functionality of the broodstock data retrieval module 512 while another software program handles another subset of the functionality of the broodstock data retrieval module 512 and the sample and broodstock data analysis module 516.

In various embodiments, the broodstock data retrieval module 512 generally represents executable instructions configured to retrieve and/or receive broodstock data corresponding to a plurality of fish individuals. With reference to FIGS. 1-4, in various embodiments, the broodstock data retrieval module 512 generates instructions for guiding or controlling operations related to collection of biological samples from a population of broodstock individuals. In some embodiments, such operations include collecting a sample of sperm from one or more broodstock males. In some embodiments, such operations include collecting a sample of ova and/or ovarian fluid from one or more broodstock females. In some embodiments, the broodstock data retrieval module 512 further generates instructions that instruct a system to perform operations including collecting of biological samples via tissue sampling of broodstock individuals. In various embodiments, tissue sampling includes collecting fin clips, muscle tissue plugs, and the like from each broodstock female 102.

In other embodiments, such as with reference to FIG. 1, instructions related to collection of biological samples includes collecting a sample of blood 104 from each broodstock female 102. In various embodiments, the broodstock data retrieval module 512 further generates instructions that instruct a system to perform operations including exposing blood from each of the broodstock females 102 to a respective blood booklet 106 including one or more layers of absorbent material (not shown) for absorbing and thereby collecting the sample of blood 104 from each brood stock female.

The broodstock data retrieval module 512 further generates instructions that instruct a system to perform operations including retrieving a normalized sampling of genetic material 108 (e.g., blood) from the blood booklet 106 associated with each individual broodstock female 102, such as by defining parameters associated with taking a similarly sized sampling from each blood booklet 106 to yield an amount of blood cells that is substantially similar from one broodstock female 102 to another. Additionally, in various embodiments, the broodstock data retrieval module 512 further generates instructions that instruct a system to perform operations including placing the collected biological samples (e.g., blood booklets 106) into cold storage after taking the initial normalized samplings of genetic material 108. Subsequently, the blood booklets 106 operate as long-term genetic material reservoirs and may be resampled for additional genetic material as needed.

In various embodiments, such as with reference to FIG. 1, the broodstock data retrieval module 512 further generates instructions that instruct a system to perform operations including labeling or otherwise tagging each broodstock individual with a unique identifier 112 provided by their respective blood booklets 106 to label biological samples (including blood, ovarian fluid, gametes, and the like) in relation to a corresponding broodstock female. For example, as illustrated in FIG. 1, such operations include tagging a first broodstock female 102(1) with a first unique identifier 112(1) from its blood booklet 106(1) and tagging a second broodstock female 102(N) with a second unique identifier 112(N) from its blood booklet 106(N). Similarly, such operations may further include tagging the second set of ova 114(N) is tagged with the second unique identifier 112(N) from the blood booklet 106(N) corresponding to the Nth broodstock female 102(N). In this manner, the broodstock data retrieval module 512 generates a connection between a blood booklet 106(N) (and its associated samplings of genetic material), the set of ova 114(N), and the broodstock female 102(N) from which the ova were extracted, thereby maintaining coordination between the various operations and constituents discussed herein.

In various embodiments, the ova storage module 514 generally represents executable instructions configured to generate instructions for guiding or controlling operations related to cold storage of gametes. With reference to FIGS. 1-4, in various embodiments, the ova storage module 514 generates instructions that instruct a system to perform operations including packaging gametes (e.g., extracted ova from block 140 of FIG. 1) into one or more storage parcels in preparation for cold storage. In various embodiments, the ova storage module 514 generates instructions that instruct a system to perform operations including splitting or otherwise allocating each of the extracted ova from a single broodstock female (e.g., the second set of ova 114(N) corresponding to the Nth broodstock female 102(N)) across two or more different storage parcels. That is, the second set of ova 114(N) is split into smaller batches.

As previously discussed with reference to FIG. 1, the parceled sets of ova 114(N) may be imaged with a light table to illuminate the interior of the ova for quality determination purposes. Further, in various embodiments, at least a first subset of the set of ova 114(N) (such as one or more of the two or more different storage parcels representing the extracted ova from a single broodstock female) may be utilized for testing purposes, such as for testing of ova quality for hatchery management purposes, metrics related to fertility of ova, and the like. Further, in various embodiments, the ova storage module 514 generates instructions that instruct a system to perform operations including retaining at least a second subset of the set of ova 114(N) for storage purposes. In various embodiments, such operations include placing the at least a second subset of the set of ova 114(N) is put into cold storage within any suitable temperature-controlled environment and storable at a controlled temperature for a predetermined duration, as previously discussed in more detail with reference to FIG. 1, to generate a time window of opportunity during which broodstock data may be applied to making a breeding decision.

In various embodiments, the sample and broodstock data analysis module 516 generally represents executable instructions configured to generate instructions for guiding or controlling operations related to characterization (including but not limited to genetically analyzing) of collected biological samples and/or gametes. With reference to FIGS. 1-4, in various embodiments, the sample and broodstock data analysis module 516 generates instructions that instruct a system to perform operations including genetically analyzing a set of DNA sequences of interest, such as characterization of one or more of genes, markers of genes, and/or marker(s) of a collection of genes. For example, in some embodiments, the sample and broodstock data analysis module 516 generates instructions that instruct a system to perform operations including characterization of one or more of genes, markers of genes, and/or marker(s) of a collection of genes with respect to relatedness 172 between individual broodstock females 102. Additionally, in some embodiments, characterization of the set of DNA sequences of interest includes detecting profiles of a set of genes in the individual one or more broodstock females 102 with respect to disease resistance 174 and further determine whether each corresponding broodstock female associated with a biological sample has a risk of transmitting a disease to offspring.

In various embodiments, the sample and broodstock data analysis module 516 generates instructions that instruct a system to perform operations including characterization of any of various genes or genomic regions of interest (including both encoding and non-encoding regions). For example, in some embodiments, the characterization of a set of DNA sequences of interest may include profile detection of a set of genes in the individual one or more broodstock females 102 indicating increased resistance to other biological issues such as parasites (e.g., sea lice), chemical issues such as antibiotic sensitivity, and the like. Further, in various embodiments, the sample and broodstock data analysis module 516 generates instructions that instruct a system to perform operations including genetically analyzing and characterization of gamete quality and progeny of broodstock individuals.

In various embodiments, the breeding decision module 518 generally represents executable instructions configured to generate instructions for guiding or controlling breeding operations based at least in part on optimizing for a breeding metric of interest. With reference to FIGS. 1-4, in various embodiments, the breeding decision module 518 receives broodstock data 170 from the sample and broodstock data analysis module 516 and generates instructions that instruct a system to perform operations including generating breeding decisions based on disease resistance determinations. Further, in various embodiments, breeding decisions (including breeding decision 180 of FIG. 1) may include, for example, mating schemes with specific male-and-female pairings, family allocation decisions, family isolation decisions, family batching decisions, gamete elimination decisions, and the like.

With reference to FIG. 3, making a breeding decision includes selecting broodstock individuals for mating based on disease resistance characteristics of the broodstock individuals and/or anticipated disease resistance characteristics of their respective families to advance an average disease resistance performance of the next generation. For example, in some embodiments, a breeding decision includes generating a mate pairing decision 302 is determined based at least in part on broodstock data 170 including genomics data with respect to biological samples (e.g., gametes, tissue samples, blood samples, and the like) collected from broodstock individuals (such as previously discussed in more detail with respect to modules 512-516). For example, in various embodiments, the family allocation decision includes assessing broodstock resistance of one or more broodstock males 304 and/or broodstock females 306 to a disease (e.g., infectious pancreas disease (IPD), infectious pancreatic necrosis (IPN), and the like). Further, in various embodiments, broodstock resistance to diseases is assessed based on the presence of putative causal single nucleotide polymorphisms (SNPs) (either positively known or hypothesized to be causally linked) associated with resistance to each of one or more diseases.

Further, in various embodiments, the breeding decision module 518 generates instructions that instruct a system to perform operations including selecting a population of sperm based on one or more calculations determined to obtain a population of offspring having a desired phenotypic makeup. In addition to the previously discussed ova-related analyses, in various embodiments, breeding decision module 518 generates instructions that instruct a system to perform operations including receiving genetic analysis (such as from module 516) indicating that a first population of sperm 204(1) from a first broodstock male 304(1) and a first blood booklet 106(1) containing blood from a first broodstock female 306(1), when paired together as breeding mates, would generate progeny individuals 310 that are all also homozygous dominant in their resistance to the given disease. Similarly, in various embodiments, breeding decision module 518 generates instructions that instruct a system to perform operations including receiving genetic analysis (such as from module 516) indicating that a second population of sperm 204(2) from a second broodstock male 304(2) and a second blood booklet 106(2) containing blood from a second broodstock female 306(2), when paired together as breeding mates, would generate a mixture of progeny including some progeny individuals 310 that are homozygous dominant, some progeny individuals 310 that are heterozygous, and/or some progeny individuals 310 that are homozygous recessive in their resistance to the given disease. Further, in various embodiments, breeding decision module 518 generates instructions that instruct a system to perform operations including receiving genetic analysis (such as from module 516) indicating that a N-th population of sperm 204(N) from a Nth broodstock male 304(N) and a N-th blood booklet 106(N) containing blood from a N-th broodstock female 306(N), when pair together as breeding mates, would generate progeny individuals 310 that are all also homozygous recessive in their resistance to the given disease.

Such sperm-related genetics data and characterization of the set of genes may be utilized by the breeding decision module 518 as the basis for ranking the broodstock individuals (or broodstock pairings) into a plurality of ranking groups, each ranking group having a different threshold or range of disease-resistance characterizations. For example, in the context of FIG. 3, the first broodstock male 304(1) or the first broodstock female 306(1) (or a mate pairing decision 302 including both the first broodstock male 304(1) and the first broodstock female 306(1)) is ranked into a first ranking group based on the broodstock individual(s) being homozygous dominant in its resistance to a given disease. Similarly, the second broodstock male 304(2) or the second broodstock female 306(2) (or a mate pairing decision 302 including both the second broodstock male 304(2) and the second broodstock female 306(2)) is ranked into a second ranking group based on the broodstock individual(s) being heterozygous in its resistance to the given disease. Further, the N-th broodstock male 304(N) or the N-th broodstock female 306(N) (or a mate pairing decision 302 including both the N-th broodstock male 304(N) and the N-th broodstock female 306(N)) being ranked into a third ranking group based on the broodstock individual(s) being homozygous recessive in its resistance to the given disease.

Accordingly, in embodiments for which optimizing for a breeding metric of interest includes optimizing for a population of offspring having a desired resistance to the given disease (i.e., one or more families having a desired phenotypic makeup), the breeding decision module 518 determines that the first ranking group having homozygous dominant broodstock individuals (or pairings of homozygous dominant broodstock individuals) is preferable over the second ranking group having heterozygous broodstock individuals (or pairings of heterozygous broodstock individuals) because mate pairings within the first ranking group have a higher probability (or in raw quantity terms, a greater number of progeny individuals) of resulting in progeny having the desired disease resistance relative to mate pairings within the second ranking group. Similarly, the breeding decision module 518 determines that the first ranking group having homozygous dominant broodstock individuals (or pairings of homozygous dominant broodstock individuals) is preferable over the third ranking group having homozygous recessive broodstock individuals (or pairings of homozygous recessive broodstock individuals) when breeding for disease resistance. Additionally, the breeding decision module 518 determines the second ranking group having heterozygous broodstock individuals (or pairings of heterozygous broodstock individuals) is also preferable over the third ranking group having homozygous recessive broodstock individuals (or pairings of homozygous recessive broodstock individuals) when breeding for disease resistance.

In another embodiment, such as described in more detail with reference to FIG. 4, the breeding decision module 518 makes breeding decisions based on analysis of progeny resulting from male-to-female pairings of breeding candidates to generate a family having a desired phenotypic makeup in the next generation. In such progeny-based analyses, the breeding decision module 518 receives genetic analysis (such as from module 516) associated with a first population of sperm from a first broodstock male 404(1a) fertilizing the first subset of the first set of ova 402(1a) from a first broodstock female (e.g., first broodstock female 102(1) of FIG. 1) to produce a first family 406(1). Such genetic analysis of the first family 406(1) evaluates genotypes of the progeny individuals 408 and determines via quantitative trait locus (QTL), SNP, and/or genetic marker analyses related to growth and/or adult size that a population of offspring arising from the breeding candidates (i.e., the first broodstock male and the first broodstock female corresponding to the first family 406(1)) would grow into an adult population at time=t having a size distribution 410 as illustrated in FIG. 4.

The breeding decision module 518 receives genetic analysis (such as from module 516) associated with a second population of sperm from the first broodstock male 404(1b) fertilizing the first subset of the second set of ova 402(2a) from a second broodstock female to produce a second family 406(2). Such genetic analysis of the second family 406(2) evaluates genotypes of the progeny individuals 408 and determines via quantitative trait locus (QTL), SNP, and/or genetic marker analyses related to growth and/or adult size that a population of offspring arising from the breeding candidates (i.e., the first broodstock male and the second broodstock female corresponding to the second family 406(2)) would grow into an adult population at time=t having a size distribution 412 as illustrated in FIG. 4. Similarly, the breeding decision module 518 receives genetic analysis (such as from module 516) associated with a first population of sperm from the second broodstock male 404(2) fertilizing the first subset of the third set of ova 402(3a) from a third broodstock female to produce a third family 406(3). Such genetic analysis of the third family 406(2) evaluates genotypes of the progeny individuals 408 and determines via quantitative trait locus (QTL), SNP, and/or genetic marker analyses related to growth and/or adult size that a population of offspring arising from the breeding candidates (i.e., the second broodstock male and the third broodstock female corresponding to the third family 406(3)) would grow into an adult population at time=t having a size distribution 414 as illustrated in FIG. 4.

Accordingly, in embodiments for which optimizing for a breeding metric of interest includes selecting mate pairings determined to give rise to families having a higher average adult weight in the next generation, the breeding decision module 518 determines selecting the first broodstock male and the first broodstock female for mating would result in a size distribution 410 that is greater (e.g., larger/heavier) relative to a size distribution 412 that would arise from a mate pairing of the same first broodstock male and the second broodstock female. Similarly, breeding decision module 518 determines selecting the second broodstock male and the third broodstock female for mating is determined to result in a size distribution 414 that is greater relative to the size distribution 412 that would arise from a mate pairing of the same first broodstock male and the second broodstock female. That is, when optimizing for the breeding metric of interest being adult size, breeding decision module 518 determines selecting the first broodstock male and the first broodstock female for mating is preferable over a pairing of the first broodstock male and the second broodstock female. Further, in various embodiments, the breeding decision module 518 is configured to generate instructions for directing fertilization of ova with one or more populations of sperm based on the analyses described herein.

In various embodiments, the allocation module 520 generally represents executable instructions configured to implement gamete and/or family allocation strategies. With reference to FIGS. 1-4, in various embodiments, the allocation module 520 receives genetic analysis (such as from module 516) and generates a family allocation decision 212 is determined based on, for example, an identification of broodstock females homozygous for a breeding metric of interest (e.g., a characteristic, phenotypic trait, and the like) that batches the resulting progeny individuals 210 into a singular incubation unit 214 for a specific customer. In other embodiments, the allocation module 520 generates the family allocation decision 212 based on, for example, genomics data from genetics analysis 130 including relatedness 172 (and therefore levels of inbreeding) between broodstock individuals to equalize (or otherwise decrease differences between) familial representation amongst multiple incubator units 214 and/or development of a pedigree-based mating strategies using broodstock data 170.

In other embodiments, the allocation module 520 receives genetic analysis (such as from module 516) and generates quarantine decisions or elimination/discard decisions. For example, with reference to FIG. 2, the allocation module 520 receives genetic analysis (such as from module 516) and generates an allocation decision in which a fourth family 208(4) produced by a broodstock female having virus-positive ovarian fluid is quarantined and separately batched into a different, second incubation unit 214(2) and therefore isolated from other incubation units 214 (such as based on detecting viral RNA indicating the presence and therefore a risk of virus transmission). In some embodiments, the allocation module 520 receives genetic analysis (such as from module 516) and generates an allocation decision in which a fifth set of ova 206(5) extracted from a broodstock female having maturation-protein-positive ovarian fluid is discarded instead of being fertilized (as the presence of maturation proteins in ovarian fluids is indicative of poor quality ova that would result in a reduction of embryo survival rates). Similarly, in various embodiments, the allocation module 520 generates similar discard decisions with respect to broodstock males, whereby poor quality sperm (e.g., having low motility) being discarded instead of being used to fertilize ova.

As described herein, computing platform(s) 502 may include electronic storage 524, one or more processors 526, and/or other components. Computing platform(s) 502 may include communication lines, or ports to enable the exchange of information with a network and/or other computing platforms. The illustration of computing platform(s) 502 in FIG. 5 is not intended to be limiting. Computing platform(s) 502 may include a plurality of hardware, software, and/or firmware components operating together to provide the functionality attributed herein to computing platform(s) 502. For example, computing platform(s) 502 may be implemented by a cloud of computing platforms operating together as computing platform(s) 502.

Electronic storage 524 may comprise non-transitory storage media that electronically stores information. The electronic storage media of electronic storage 524 may include one or both of system storage that is provided integrally (i.e., substantially non-removable) with computing platform(s) 502 and/or removable storage that is removably connectable to computing platform(s) 502 via, for example, a port (e.g., a USB port, a firewire port, etc.) or a drive (e.g., a disk drive, etc.). Electronic storage 524 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 524 may include one or more virtual storage resources (e.g., cloud storage, a virtual private network, and/or other virtual storage resources). Electronic storage 524 may store software algorithms, information determined by processor(s) 526, information received from computing platform(s) 502, information received from remote platform(s) 504, and/or other information that enables computing platform(s) 502 to function as described herein.

Processor(s) 526 may be configured to provide information processing capabilities in computing platform(s) 502. As such, processor(s) 526 may include one or more of a digital processor, an analog processor, a digital circuit designed to process information, an analog circuit designed to process information, a state machine, and/or other mechanisms for electronically processing information. Although processor(s) 526 is shown in FIG. 5 as a single entity, this is for illustrative purposes only. In some implementations, processor(s) 526 may include a plurality of processing units. These processing units may be physically located within the same device, or processor(s) 526 may represent processing functionality of a plurality of devices operating in coordination. Processor(s) 526 may be configured to execute modules 512, 514, 516, 518, and/or 520, and/or other modules. Processor(s) 526 may be configured to execute modules 512, 514, 516, 518, and/or 520, and/or other modules by software; hardware; firmware; some combination of software, hardware, and/or firmware; and/or other mechanisms for configuring processing capabilities on processor(s) 526. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 512, 514, 516, 518, and/or 520 are illustrated in FIG. 5 as being implemented within a single processing unit, in implementations in which processor(s) 526 includes multiple processing units, one or more of modules 512, 514, 516, 518, and/or 520 may be implemented remotely from the other modules. The description of the functionality provided by the different modules 512, 514, 516, 518, and/or 520 described herein is for illustrative purposes, and is not intended to be limiting, as any of modules 512, 514, 516, 518, and/or 520 may provide more or less functionality than is described. For example, one or more of modules 512, 514, 516, 518, and/or 520 may be eliminated, and some or all of its functionality may be provided by other ones of modules 512, 514, 516, 518, and/or 520. As another example, processor(s) 526 may be configured to execute one or more additional modules that may perform some or all of the functionality attributed herein to one of modules 512, 514, 516, 518, and/or 520.

Those skilled in the art will understand that although primarily discussed here in the context of gamete storage and genetic analysis for making breeding decisions, in various embodiments, the concepts discussed herein may similarly be applied to cold storage of biomaterial at any period of an organism's life cycle without departing from the scope of this disclosure. For example, in various embodiments, such biomaterial is sourced from stages starting from a single-celled zygote after fertilization and onwards, including cleavage periods, blastula periods, hatching periods, larval periods, fetal periods (in embodiments utilizing, for example, agricultural animals), and the like.

Growth has typically been a key driver in aquaculture, but now other factors such as disease resistance are becoming increasingly important. Genomic selection provides various tools to develop more resilient and stronger animals; thus it has a major impact on animal welfare as well. To improve the results of aquaculture breeding, FIGS. 1-5 describe techniques for prolongation of viable gamete retention and selection of populations of broodstock individuals for use in making breeding decisions to improve conception rates and qualities of artificially bred animals. In the context of aquaculture, fish in captivity may not always reproduce at the most advantageous times and therefore alteration of the spawning cycle is often desirable. Alteration of spawning cycles allows a farmer to obtain fish outside of the normal spawning season, such as to lengthen time for grow-out or to produce hybrids with other species.

Conventional selective breeding techniques, especially with particular species within aquaculture industries, have sometimes been less successful and more limited in their time window of applicability relative to terrestrial livestock due to rapid degradation of gamete quality (e.g., viability, motility, rates of fertilization, and the like) after spawning. Breeding programs themselves are additionally limited by the coordinated reproduction of individual generations, which if not fulfilled results in rapid degradation of gametes whether spawned or not. To alleviate problems associated with gamete degradation, the reproduction timing manipulation and gamete cold storage techniques described herein better aligns periods of sperm viability with the fertile life of ova and improves the precision of fertilization timing windows. During periods within which gamete quality is preserved while under cold storage (e.g., prolongation of viable sperm and ova retention), multiple breeding decisions may be determined to add data connectivity and additional value to the underlying gametes.

The prolongation of viable sperm and ova retention via cold storage techniques increases the length of a time window of opportunity between gamete collection (e.g., from spawning, manual stripping, and the like) and a subsequent point in time at which the gametes begin appreciably degrading (e.g., loss in fertility). The increased time window of opportunity allows time for genetic testing and analyses that would otherwise not be completed before germplasm degradation, thereby allowing time to perform additional optimizations for selective breeding decisions that are beyond what is currently possible due to time constraints. That is, the increased time window of opportunity resulting from cold storage of ova makes available additional testing methods and/or breeding decisions (genetics-based or otherwise).

In the context of genetics, genomic selection not only offers increased selection accuracy in general, but also opens for increased selection intensity for sib traits. Sib traits are traits that with conventional selection methods limit selection to amongst families and includes traits such disease resistance and carcass quality. With implementation of genomic information, not only may the best families be determined but the best breeding candidates within those families may also be identified. As production on genetically improved stocks is more biologically efficient than production on non-improved stocks, well-designed selection approaches can, for example, speed up a population's adaptation to diseases and the natural environment (amongst various factors), in a manner similar to natural selection but faster. In this manner, the various cold-storage-enabled biological samples analyses and progeny analyses described herein utilize the natural genetic variation provided by nature and capitalize upon it in an improved manner through obtaining more accurate information about the genetic constitution of breeding candidates and more accurately defining the genetic potential of breeding candidates (e.g., broodstock individuals).

Further, the various cold-storage-enabled biological samples analyses and progeny analyses described herein allow for collection of gametes and various screenings prior to artificial fertilization. For example, screening tests related to gametes may inform decisions such as discarding (or quarantine, segregate, treat, or otherwise artificially intervene) of bad quality gametes, quarantining gametes from diseased broodstock individuals, prevent embryonic losses due to poorly matched mating pairs that give rise to low quality offspring, thereby improving conception rates and quality of artificially-bred animals. In this manner, various issues related to disease-prone, low yields, and other growing issues may be improved.

In some embodiments, certain aspects of the techniques described above may implemented by one or more processors of a processing system executing software. The software includes one or more sets of executable instructions stored or otherwise tangibly embodied on a non-transitory computer readable storage medium. A computer readable storage medium may include any non-transitory storage medium, or combination of non-transitory storage media, accessible by a computer system during use to provide instructions and/or data to the computer system. Such storage media can include, but is not limited to, optical media (e.g., compact disc (CD), digital versatile disc (DVD), Blu-Ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)).

The software can include the instructions and certain data that, when executed by the one or more processors, manipulate the one or more processors to perform one or more aspects of the techniques described above. The non-transitory computer readable storage medium can include, for example, a magnetic or optical disk storage device, solid state storage devices such as Flash memory, a cache, random access memory (RAM) or other non-volatile memory device or devices, and the like. The executable instructions stored on the non-transitory computer readable storage medium may be in source code, assembly language code, object code, or other instruction format that is interpreted or otherwise executable by one or more processors.

Further, in some embodiments, the non-transitory computer readable medium store a set of instructions, which when executed by a computing device, establishes a set of computer processes. As described herein, a “computer process” is the performance of a described function in a computer using computer hardware (such as a processor, field-programmable gate array or other electronic combinatorial logic, or similar device), which may be operating under control of software or firmware or a combination of any of these or operating outside control of any of the foregoing. All or part of the described function may be performed by active or passive electronic components, such as transistors or resistors. In using the term “computer process” we do not necessarily require a schedulable entity, or operation of a computer program or a part thereof, although, in some embodiments, a computer process may be implemented by such a schedulable entity, or operation of a computer program or a part thereof. Furthermore, unless the context otherwise requires, a “process” may be implemented using more than one processor or more than one (single- or multi-processor) computer.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

What is claimed is:

1. A method of producing a population of fertilized ova, the method comprising:
obtaining and cold storing a plurality of sets of fish ova, each set of the plurality of sets of fish ova being extracted from a corresponding broodstock female, wherein cold storing includes labeling each set of fish ova in relation to the corresponding broodstock female;
collecting a biological sample from each broodstock female;
with respect to each broodstock female associated with a corresponding set of fish ova and biological sample, after the corresponding set of fish ova has been extracted, obtaining broodstock data that includes (i) characterization of a set of DNA sequences of interest based at least in part on the collected biological sample;
selecting, based at least in part on the broodstock data, a first subset of the plurality of sets of fish ova for fertilization; and
selecting, based on a determination of optimizing for a breeding metric of interest, a population of sperm to fertilize the first subset of the plurality of sets of fish ova.

2. The method of claim 1, wherein cold storing the plurality of sets of fish ova comprises:
placing at least a subset of the plurality of sets of fish ova in a refrigerated environment at a temperature substantially proximal to a freezing point of water without causing freezing damage to the subset of the plurality of sets of fish ova.

3. The method of claim 1, wherein the determination of optimizing for the breeding metric of interest includes:
a determination of whether each broodstock female corresponding to the first subset of the plurality of sets of fish ova has a risk of transmitting a disease to offspring.

4. The method of claim 1, wherein the determination of optimizing for the breeding metric of interest includes:
a determination of allocating progeny resulting from fertilizing ova of two or more broodstock females to a single incubation unit.

5. The method of claim 1, wherein the selecting the population of sperm further includes:
selecting the population of sperm based on one or more calculations determined to obtain a population of offspring having a desired phenotypic makeup.

6. The method of claim 1, further comprising:
fertilizing the first subset of the plurality of sets of fish ova with the population of sperm.

7. The method of claim 6, further comprising:
genetically analyzing genotypes of progeny resulting from fertilizing the first subset of the plurality of sets of fish ova prior to fertilizing a second subset of the plurality of sets of fish ova.

8. The method of claim 1, wherein collecting the biological sample from each broodstock female further comprises:
collecting a sample of blood from each broodstock female via a blood booklet.

9. A method, comprising:
receiving broodstock data corresponding to a plurality of sets of fish ova, wherein the broodstock data includes data labeling which associates each of the plurality of sets of fish ova in relation to a corresponding broodstock female and a biological sample collected from the corresponding broodstock female, each set of fish ova having been extracted from the corresponding broodstock female and placed into cold storage;
selecting, based at least in part on genetic analysis of the biological sample and the broodstock data, a first subset of the plurality of sets of fish ova for fertilization; and
selecting, based on a determination of optimizing for a breeding metric of interest, a population of sperm to fertilize the first subset of the plurality of sets of fish ova.

10. The method of claim 9, further comprising:
genetically analyzing the biological sample collected from and associated with the corresponding broodstock female;

11. The method of claim 10, wherein genetically analyzing the biological sample comprises:
characterizing a set of DNA sequences of interest based at least in part on the biological sample.

12. The method of claim 9, further comprising:
determining whether the corresponding broodstock female associated with the biological sample has a risk of transmitting a disease to offspring.

13. The method of claim 9, further comprising:
genetically analyzing genotypes of progeny resulting from fertilizing the first subset of the plurality of sets of fish ova prior to fertilizing a second subset of the plurality of sets of fish ova with the population of sperm.

14. The method of claim 9, further comprising:
allocating progeny resulting from fertilizing ova of two or more broodstock females to a single incubation unit.

15. The method of claim 9, wherein the selecting the population of sperm further includes:
selecting the population of sperm based on one or more calculations determined to obtain a population of offspring having a desired phenotypic makeup.

16. A non-transitory computer readable medium embodying a set of executable instructions, the set of executable instructions to manipulate at least one processor to:
receive broodstock data corresponding to a plurality of sets of fish ova, wherein the broodstock data includes data labeling which associates each of the plurality of sets of fish ova in relation to a corresponding broodstock female and a biological sample collected from the corresponding broodstock female, each set of fish ova having been extracted from a corresponding broodstock female and placed into cold storage;
select, based at least in part on genetic analysis of the biological sample and the broodstock data, a first subset of the plurality of sets of fish ova for fertilization; and
select, based on a determination of optimizing for a breeding metric of interest, a population of sperm to fertilize the first subset of the plurality of sets of fish ova.

17. The non-transitory computer readable medium of claim 16, further embodying executable instructions to manipulate at least one processor to:
characterize a set of DNA sequences of interest based at least in part on the biological sample.

18. The non-transitory computer readable medium of claim 16, further embodying executable instructions to manipulate at least one processor to:
determine whether the corresponding broodstock female associated with the biological sample has a risk of transmitting a disease to offspring.

19. The non-transitory computer readable medium of claim 16, further embodying executable instructions to manipulate at least one processor to:
genetically analyze genotypes of progeny resulting from fertilizing the first subset of the plurality of sets of fish ova with the population of sperm.

20. The non-transitory computer readable medium of claim 16, further embodying executable instructions to manipulate at least one processor to:
select the population of sperm based on one or more calculations determined to obtain a population of offspring having a desired phenotypic makeup.
